(19) United States
(12) Patent Application Publication
(10) Pub. No.: US 2025/0251503 A1
(43) Pub. Date: Aug. 7, 2025

(54) Acoustic Subwavelength Imaging System

(71) Applicant: Wisconsin Alumni Research Foundation, Madison, WI (US)

(72) Inventors: Chu Ma, Middleton, WI (US); Jinuan Lin, Madison, WI (US)

(21) Appl. No.: 18/435,421

(22) Filed: Feb. 7, 2024

(51) Int. Cl. G01S 7/52; A61B 8/08

(52) U.S. Cl. CPC G01S 7/52079; A61B 8/0891; A61B 8/481

(57) ABSTRACT

An acoustic imaging system employs a set of subwavelength scatterers in the vicinity of the imaged object and having unknown locations to obtain multiple acoustic images with the scatterers in different positions, the multiple acoustic images processed using joint sparsity assumptions to provide an image with subwavelength resolution.

### STATEMENT REGARDING FEDERALLY SPONSORED RESEARCH OR DEVELOPMENT

This invention was made with government support under 2237619 awarded by the National Science Foundation. The government has certain rights in the invention.

### CROSS REFERENCE TO RELATED APPLICATION

--

### BACKGROUND OF THE INVENTION

The present invention relates generally to acoustic imaging systems and in particular to an acoustic imaging system that can resolve object features substantially smaller than the point spread function of the imaging system.

Acoustic imaging plays a crucial role in various fields, including underwater sonar, nondestructive testing, and biomedical imaging. Ultrasound imaging, in particular, is widely used in medical diagnoses due to its non-invasive nature and real-time imaging capability.

The resolution of conventional acoustic imaging systems is inherently constrained by the diffraction limit being a function of the wavelength of the acoustic signal, a fundamental physical limitation to all wave-based imaging technologies, such as in optical, microwave, thermal, photoacoustic, and acoustic imaging. While shorter wavelength acoustic signals produce better resolution, they typically suffer from shorter penetration depth. Therefore, there has been significant interest in developing far-field subwavelength imaging techniques.

Existing subwavelength acoustic imaging systems have employed meta-materials deposited close to the object to be imaged in known locations during imaging. The requirement of placing structures close to the object to be imaged, either in known locations or in a way to allow measuring the locations of the structures during imaging, limits practical application of these techniques, particularly for biomedical imaging where the objects being imaged have limited accessibility.

### SUMMARY OF THE INVENTION

The present invention provides a subwavelength acoustic imaging system that employs “blind” scatterers, that is scattering structure having a changing unknown position. This makes the imaging technique practical, for example, for biomedical imaging, where the scatterers can be microbubbles or the like injected into a blood vessel to be imaged.

In one embodiment, the invention provides an acoustic imaging device for subwavelength imaging of an object within a set of scattering elements with subwavelength dimensions. The imaging device includes at least one acoustic transducer capturing an acoustic image of the object at an acoustic wavelength and an image processor executing a stored program to: (a) acquire multiple acoustic images of the object with the set of scattering elements in different unknown locations in different images; and (b) process the multiple acoustic images to provide a higher resolution acoustic image revealing subwavelength features of the object. It is thus a feature of at least one embodiment of the invention to provide

subwavelength imaging at a far field location using scatterers and without precise knowledge of scatterer location. This ability to image without precise knowledge of the scatterer location allows imaging in areas that are difficult to access such as blood vessels using flowing microbubbles.

The processing may iteratively model the imaging process to match the model to the received multiple acoustic images.

It is thus a feature of at least one embodiment of the invention to accommodate the missing information about scatterer location by employing an iterative process.

The processing may implement a joint sparsity calculation using the point spread function as a parameter.

It is thus a feature of at least one embodiment of the invention to provide for reconstruction when sparsity in an object image can be assumed.

The processing may, before reconstruction, backproject the acquired multiple acoustic images to an object plane of the object before applying the joint sparsity calculation.

It is thus a feature of at least one embodiment of the invention to remove the effects of imaging distance from the calculation.

The imaged object and set of scattering elements maybe surrounded by a liquid.

It is thus a feature of at least one embodiment of the invention to provide an imaging technique well suited for acoustic imaging where scatterers can be suspended in a flowing liquid. In one embodiment, the scattering elements may be microbubbles in the liquid.

It is thus a feature of at least one embodiment of the invention to provide a system that can work with biocompatible scatterers such as microbubbles.

In some embodiments, the scattering elements may be composite materials presenting multiple concentric layers of material that provide resonance at the ultrasound frequency.

It is thus a feature of at least one embodiment of the invention to provide improved scatterers use with this system.

These particular objects and advantages may apply to only some embodiments falling within the claims and thus do not define the scope of the invention.

### BRIEF DESCRIPTION OF THE DRAWINGS

FIG. 1 is a simplified diagram of an ultrasonic imaging system useful for practice of the present invention and providing an ultrasonic transducer position to image structure in a blood vessel through which microbubble scatterers (enlarged in inset) are dispersed;

FIG. 2 is a plot showing example subwavelength image data that may be produced by the ultrasonic imaging system of FIG. 1 and showing a comparison to the point spread function of the ultrasonic imaging probe; and

FIG. 3 is a flowchart of a program executable on the ultrasonic imaging machine of FIG. 1 to produce the subwavelength image data of FIG. 2.

### DETAILED DESCRIPTION OF THE PREFERRED EMBODIMENT

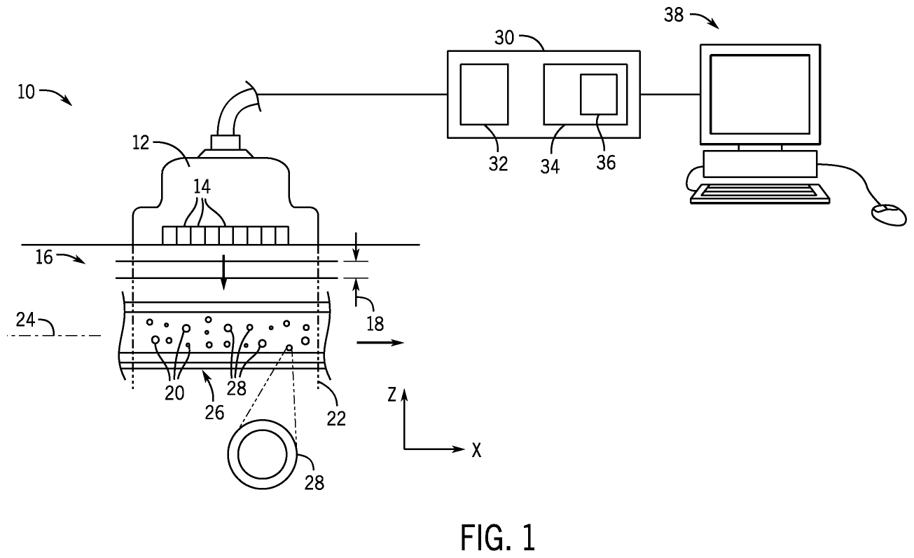

Referring now to FIG. 1, an acoustic imaging machine 10 implementing one embodiment of the invention, may provide an ultrasound transducer 12 having an array of transducer elements 14 that may be excited to generate a stimulating acoustic wave 16 having a wavelength 18. The acoustic wave 16 may be directed toward an object 20 to be imaged, for example, a blood vessel 22 and calcification within a blood vessel 22.

The acoustic wave 16 may pass from the transducer elements 14 to the object 20 through the body which may be regarded as a substantially continuous acoustic media of liquid water. The object 20 may lie in an object plane 24 that will typically, for example, be many times the wavelength 18 from the transducers 12. In this respect the transducer 12 detects a far field acoustic signal and may be practically located one or more centimeters away from the image plane. Such a distance is possible with penetration depths achievable with an acoustic wave 16 at 3 MHz and more generally from 2 to 12 MHz.

The ultrasound transducer 12 will have a spatial resolution determined in part by the overall size of the transducer 14 and the wavelength. This resolution defines a point spread function, in one example, having a full width half maximum (FWHM) of approximately 1.2 mm along the x-axis perpendicular to a propagation direction (z-axis) of the acoustic wave 16.

During imaging, a region 26 near the object 20, for example, a volume of the blood vessel 22 within the image area of the transducer 12, holds a set of suspended subwavelength scatterers 28, for example, microbubbles or glass microspheres. Microbubbles suitable for this purpose may be generated according to techniques known in the art from small spheres of gas of low solubility (e.g., perfluorocarbon) in the surrounding liquid stabilized with a thin biocompatible encapsulation layer such as a phospholipid monolayer.

Practical scatterer sizes range from 30 μm to 90 μm for a typical ultrasound system. The scatterers 28 will have a largest dimension, for example, a diameter for spherical shapes, of less than ⅕ the wavelength 18 and typically less than 1/10 the wavelength 18.

Importantly, the scatterers 28 have acoustic properties sufficiently different from that of the surrounding fluid of water so as to substantially reflect and/or scatterer the acoustic wave.

In some embodiments, the scatterers 28 may have a gradient of acoustic properties obtained, for example, with multiple concentric layers of material that provide resonance at the frequency of the wave emitted by the ultrasound transducer 28.

Generally, these scatterers 28 will be in motion, for example, moving with flow of the blood of a blood vessel and randomly dispersed. Nevertheless the scatterers 28 may have a generally defined density within the liquid described by a filling ratio being the ratio of the total area of the scatterers 28 to the total area of the region in which they are constrained, the latter generally being a region of interest around the object 20 in an imaging plane within the image. Filling ratios from 10% to 50% are contemplated with filling ratios of 20% being preferred. The filling ratio implements a trade-off between imaging speed and quality with relatively low scatterer concentrations requiring additional measurements as will be discussed below but producing better image quality. These fill ratios may include glass microsphere concentrations of about 1%.

Referring still to FIG. 1, acoustic wave 16 from the transducer 12 will arrive at and reflect from the object 20 to be scattered by the scatterers 28 on the return path producing spatial mixing that converts the evanescent components in the waves from the object 20 to propagating components that can reach the far field detector of the transducer 12. These propagating components are received by the transducer elements 14 operating to generate corresponding electrical signals from which image phase and amplitude values at different points along the transducer surface are obtained.

This image data is then transmitted to a processing system 30 to be further processed to produce of an image, for example, a B mode image, or other image data output. The processing system 30 will generally include one or more computer processors 32 and a memory 34 holding a stored program 36 as will be described. The processing system 30 may communicate with a user interface terminal system 38 providing a graphic screen, keyboard, mouse, or the like as is generally understood in the art and through which data may be entered into the processing system 30 or output from the processing system 30, for example, in the form of an image or quantitative data representations of image data.

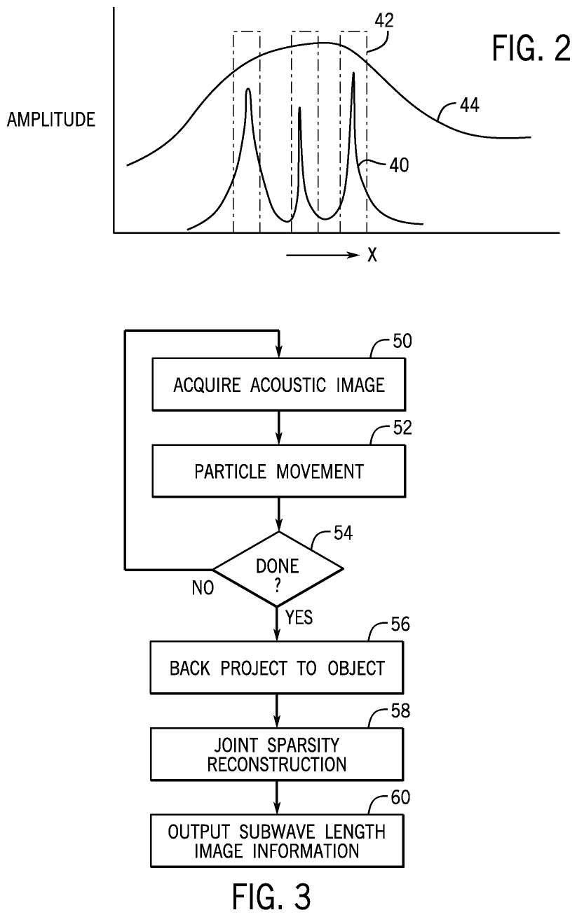

Referring now to FIG. 2, processing of the data received from the transducer 12 by the processing system 30, per a simulation conducted by the inventors, may provide an image 40, for example, shown along the x-axis indicating three peaks representing the three imaged objects 20, for example, having a separation of 0.1 mm (e.g., approximately 0.2 times the wavelength of the acoustic wave 16) and a diameter of 10 μm. The objects 20 are depicted by the ground truth data 42 and are generally sparse spatially. In this example, the wavelength 18 of the acoustic wave 16 will be approximately 0.5 mm (3 MHz) and the image 40 will provide a subwavelength resolution of over 10 times that of the wavelength 18 beyond that expected to be possible given the point spread function 44 with a FWHM of 1.2 mm. The size of the scatterers 28 is 10 u with a filling ratio of 10%. The transducer 12 is separated from the object plane 24 by 25 mm and 50 measurements were assumed. Similar physical experiments validate these conclusions.

Referring now to FIGS. 1 and 3, this subwavelength image 40 is obtained using the system of FIG. 1 executing program 36 to acquire multiple acoustic images (representing snapshots in time of received phase and amplitude information at the transducer 12) per process block 50. Each of these images 40 is separated by a time during which there is intervening particle motion per process block 52. Significantly, the exact positions of the scatterers 28 are not known or measured.

At decision block 54, after a predetermined number of images 40 have been obtained (pre-determined by a total amount of acquisition time considered acceptable and the filling ratio as discussed above), each image 40 may be back projected per process block 56 to the object plane 24. Back projection re-creates the image data at that object plane 24 such as would produce the received far field signal at the transducer 12 under the assumption of a uniform liquid media without scatterers 28.

Each of these images 40 is then combined at process block 58 to deduce a shape of the image object 20 consistent with the back projected images 40. This reconstruction uses the point spread function of the imaging system and may, for example, implement a joint sparsity reconstruction iteratively identifying a shape of objects 20 that would produce the back projected image data under an assumption that the object is spatially sparse.

In this system, the scattering of the acoustic wave 16 by the scatterers 28 provides structured illumination for the object. The image data collected at process block 50 yₘ(x) can be described by:

$${y}_{m} ( x ) = h ( x ) * [ {i}_{m} ( x ) \cdot o ( x ) + {i}_{m} ( x ) ] + {ϵ}_{m} \qquad ( 1 )$$

$$for m = 1 , … , M$$

where:
h(x) is the point spread function 44;
o(x) is the spatial distribution of the object 20;
ϵₘ is noise;
iₘ(x) is the distribution of scatterers 28; and
M are the different distribution patterns of the images 40 collected.

Here, the collected image data yₘ(x) contain not only the reflected signals from the object 20 but also the reflected signals from the scatterers 28 represented by the term h(x)*iₘ(x). Since the scatterers 28 distribute evenly and randomly, the extra term h(x)*iₘ(x) can be augmented to the noise term ϵₘ. In k-space, the product iₘ(x)·o(x) becomes a convolution as shown by:

$${Y}_{m} ( {k}_{x} ) = H ( {k}_{x} ) \cdot [ {I}_{m} ( {k}_{x} ) * O ( {k}_{x} ) ] + C \qquad ( 2 )$$

$$for m = 1 , … , M$$

Since the iₘ(x) contains higher spatial frequencies than the point spread function h(x), the convolution Iₘ(kₓ)*O(kₓ) enables parts of the high-frequency spectrum of the object 20 to be shifted down to the passband of the point spread function 44. Thus, the evanescent-wave components of the object 20 can be encoded into propagating waves and reach the far field location of the transducer 12.

Since the illumination patterns iₘ(x) are unknown, the system (1) is underdetermined, and solving for the object distribution o(x) is an ill-posed linear inversion problem. Therefore, additional constraints are required, such as all illumination patterns adding up to a uniform pattern or sparse object distribution. See generally, E. Mudry, K. Belkebir, J. Girard, J. Savatier, E. Le Moal, C. Nicoletti, M. Allain, and A. Sentenac, “Structured illumination microscopy using unknown speckle patterns,” Nature Photonics, vol. 6, no. 5, pp. 312-315, 2012 and T. W. Murray, M. Haltmeier, T. Berer, E. Leiss-Holzinger, and P. Burgholzer, “Super-resolution pho-to acoustic microscopy using blind structured illumination,” Optica, vol. 4, no. 1, pp. 17-22, 2017, hereby incorporated by reference.

In the present invention, a joint-sparsity constraint maybe employed that explores the common features possessed by multiple measurements of the same object. By solving the following optimization problem, including iterative least squares with a joint-sparsity penalty, the object distribution o(x) can be reconstructed from M measurements and the system's point spread function 44:

$${min}_{u} \frac{1}{2} {\sum }_{m = 1}^{M} {\sum }_{i = 1}^{N} {\vert h * {u}_{m} ( {x}_{i} ) - {y}_{m} ( {x}_{i} ) \vert}^{2} + {α}_{1} {‖ u ‖}_{2 , 1} + \frac{{α}_{2}}{2} {‖ u ‖}_{2}^{2} \qquad ( 3 )$$

where:
uₘ(xᵢ) is the image field distribution at the object plane 24
h, uₘ, yₘ∈R^{N}.

In the cost function, the l_{2,1}-norm ∥u∥_{2,1}=Σₘ₌₁^{M}|uₘ(xᵢ)|²)^{1/2} is the joint-sparsity term with regularization parameter α₁, and ∥u∥₂²=Σₘ₌₁^{M}Σᵢ₌₁^{N}|uₘ(xᵢ)|² provides improved stability with regularization parameter α₂. The l_{2,1}-norm first takes the l₂-norm of all sampled signals at each spatial sample location (Σₘ₌₁^{M}|uₘ(xᵢ)|²)^{1/2}. Since the object distribution is invariant among all M image measurements, only certain spatial sample locations among all {xi}'s would have consistently significant values uₘ(xᵢ) jointly for all image measurements and consequently yield significant values of the l₂-norm. Then the l₁-norm is taken with respect to the l₂-norm at all the spatial locations, which enforces sparsity of the object's spatial distribution jointly among all image measurements. The block-FISTA algorithm is used to solve (3). See T. W. Murray, M. Haltmeier, T. Berer, E. Leiss-Holzinger, and P. Burgholzer, “Super-resolution photoacoustic microscopy using blind structured illumination,” Optica, vol. 4, no. 1, pp. 17-22, 2017. [13] hereby incorporated by reference.

Additional details about the use of the joint-sparsity constraint are provided by Duarte, Marco & Sarvotham, S & Wakin, M & Baron, Dror & Baraniuk, Richard. (2005). Joint sparsity models for distributed compressed sensing. Online Proceedings of the Workshop on Signal Processing with Adaptive Sparse Structured Representations (SPARS), hereby incorporated by reference.

Referring still to FIG. 3, at process block 60 output is provided providing data from the subwavelength image, for example, in the form of an image, a plot as shown in FIG. 2, or quantitative data as may be desired.

It will be appreciated that this technique is not limited to medical applications but can be used for subwavelength image data acquisition in a variety of applications. Importantly, the invention can implement a flexible trade-off between the penetration depth of the acoustic signal and the resolution, allowing lower frequencies to be used for greater penetration and subwavelength image data acquired to obtain standard resolutions at those greater depths. More generally, the scatterers 28 need not be small but simply subwavelength with respect to the interrogating sound, for example, permitting moving plants or schools of fish to behave like scatterers in underwater imaging or the like.

The terms image and acoustic image refer generally to spatially linked data, for example, in an area to produce a conventional image but also including one-dimensional images and quantitative data reflecting information that would be obtained in an image.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

What we claim is:

1. An acoustic imaging device for subwavelength imaging of an imaged object within a set of scattering elements with subwavelength dimension and movably positioned around the imaged object, the device comprising:
at least one acoustic transducer capturing an acoustic image of the imaged object and scatterers at an acoustic wavelength, the acoustic transducer having a point spread function; and
an image processor executing a stored program to:
(a) acquire multiple acoustic images of the imaged object with the set of scattering elements in different unknown locations in different images; and
(b) process the multiple acoustic images using the point spread function in a predefined assumption of sparsity of the imaged object to provide an acoustic image revealing subwavelength features of the imaged object smaller than the acoustic wavelength.

2. The acoustic imaging device of claim 1 wherein the image processor executes the stored program to iteratively model the imaged object with subwavelength dimensions to match the model to the received multiple acoustic images.

3. The acoustic imaging device of claim 2 wherein the processing employs a joint sparsity calculation using the point spread function as a parameter.

4. The acoustic imaging device of claim 3 wherein the processing back projects the acquired multiple acoustic images to an object plane of the imaged object before applying the joint sparsity calculation.

5. The acoustic imaging device of claim 1 wherein the imaged object and set of scattering elements are surrounded by a liquid.

6. The acoustic imaging device of claim 5 further including microbubble scattering elements in the liquid.

7. The acoustic imaging device of claim 5 further including composite materials scattering elements presenting concentric layers of material providing a resonance at the frequency of the acoustic wave from the transducer.

8. The acoustic imaging device of claim 1 wherein the scatterers have an average cross-sectional dimension less than ⅕ of the acoustic wavelength.

9. The acoustic imaging device of claim 1 wherein the ratio of a total area of the scattering elements in a predetermined region of the acquired images of the image object constraining the scattering elements is less than 30%.

10. The acoustic imaging device of claim 1 wherein the at least one acoustic transducer provides multiple transducer elements providing an output acoustic wave directed at the imaged object and measuring phase and acoustic amplitude of a return acoustic wave at a variety of locations to generate the acoustic image.

11. The acoustic imaging device of claim 1 wherein at least one ultrasonic transducer is an ultrasonic transducer.

12. A method of acoustic imaging providing for subwavelength imaging of an imaged object within a set of scattering elements with subwavelength dimension and movably positioned around the imaged object, the method comprising:
(a) using at least one acoustic transducer to capture multiple acoustic images of the imaged object and scatterers at an acoustic wavelength, the acoustic transducer having a point spread function with the set of scattering elements in different unknown locations in different images; and
(b) processing the multiple acoustic images using the point spread function in a predefined assumption of sparsity of the imaged object to provide an acoustic image revealing subwavelength features of the imaged object smaller than the acoustic wavelength.

13. The method of claim 12 wherein the processing of the multiple acoustic images iteratively models the imaged object with subwavelength dimensions to match the model to the received multiple acoustic images.

14. The method of claim 13 wherein the processing of the multiple acoustic images employs a joint sparsity calculation using the point spread function as a parameter.

15. The method of claim 14 wherein the processing back projects the acquired multiple acoustic images to an object plane of the imaged object before applying the joint sparsity calculation.

16. The method of claim 12 wherein the imaged object and set of scattering elements are surrounded by a liquid.

17. The method of claim 16 further where in the scattering elements are microbubbles of gas in the liquid.

18. The method of claim 16 wherein the scattering elements are formed of composite materials presenting a gradient of acoustic properties along a path from a center of a scattering element to its outer periphery that provides resonance at the frequency of the acoustic wave from the transducer.

19. The method of claim 12 wherein the scatterers have an average cross-sectional dimension less than ⅕ of the acoustic wavelength.

20. The method of claim 12 wherein the ratio of a total area of the scattering elements in a predetermined region of the acquired images of the image object constraining the scattering elements is less than 30%.
